(19) United States
(12) Patent Application Publication
(10) Pub. No.: US 2022/0143244 A1
(43) Pub. Date: May 12, 2022

(54) METHOD AND DEVICE FOR DISINFECTING CLEAN ROOMS

(71) Applicant: GfPS- GESELLSCHAFT FÜR PRODUKTIONSHYGIENE UND STERILITÄTSSICHERUNG MBH, Aachen (DE)

(72) Inventor: Simon DIETZ, Aachen (DE)

(21) Appl. No.: 17/599,988

(22) PCT Filed: Apr. 1, 2020
(86) PCT No.: PCT/DE2020/100268

§ 371 Date: Sep. 29, 2021

(30) Foreign Application Priority Data
Apr. 1, 2019 (DE) 10 2019 108 397.4

(51) Int. Cl. A61L 2/18; A61L 2/22; A61L 2/24; A61L 2/26; B64C 39/02; B64D 1/18

(52) U.S. Cl. CPC A61L 2/18; A61L 2/22; A61L 2/24; A61L 2/26; B64C 39/024; B64D 1/18; B64C 2201/127; A61L 2202/14; A61L 2202/15; A61L 2202/16; A61L 2202/25; B64C 2201/123; A61L 2202/11

(57) ABSTRACT

The invention relates to a method of disinfecting clean rooms for administration of a disinfectant, which is sprayed onto the surfaces to be disinfected by means of an unmanned aerial vehicle from the air; the disinfectant being stored on board the aerial vehicle in at least one tank. The invention further relates to a device for performing this method, comprising an accordingly adapted unmanned aerial vehicle.

The invention relates to a method of disinfecting clean rooms by administering a disinfectant by means of an unmanned aerial vehicle. The invention further relates to a device for performing this method, comprising a correspondingly designed unmanned aerial vehicle.

Hygiene and cleanliness of clean rooms play a central role for ensuring product quality. Clean rooms exist both in biological areas, such as pharmaceutics, reproductive medicine, cosmetics, food industry or biological labs, and anorganic areas, such as semiconductor production, microelectronics, micromechanics, optics, satellite technology or surface coating technology.

In many areas, for example in pharmaceutics, clean room conditions are legally required by predefined standards. Also, there are fields such as semiconductor production, in which clean room technology is essential for increasing the production quality and reducing scrap.

Thus, the cleaning and disinfection of surfaces represent important steps for maintaining cleanliness in pharmaceutical production. For example, chapter 797 of the US Pharmacopoeia (USP) requires the following of strict processes in manufacturing pharmaceutical products. One measure for achieving microbial control within a clean room is the use of defined cleaning techniques for walls, ceilings, floors and surfaces at working height, as well as the use of suitable cleaning and disinfecting agents.

In the face of increased occurrence of multiresistant germs, effective disinfection is of decisive importance.

In the state of the art, clean rooms are cleaned and disinfected manually. For this purpose, staff must be subjected to qualified training in order to apply the disinfectant on the surfaces of rooms to be disinfected in a controlled manner. Homogeneous moistening is essential for the disinfectant's effect. The agent must moisten the entire surface evenly over a certain minimum time; otherwise, the effective power is not achieved.

The disinfecting process is made more difficult by the fact that the microbiological particulate impurities on the surfaces are not visible to the human eye. The staff member can neither recognize contaminated areas nor visually control the success of his disinfecting activity.

Another challenge consists in the fact that success monitoring only takes place on a random basis since an audit trail (100% control) for the process is not yet possible. It is true that in many cases, the life science industry uses surveillance cameras; however, they allow only an indirect control of successful and even disinfection. Increasingly, external cleaning contractors are made use of for disinfecting the surfaces; in this case, success monitoring is even more necessary. Here, audits regularly show deviations in the handling of the disinfectant and in the complete disinfection of all surfaces as required. Many staff members of temporary work agency are not sufficiently aware of the invisible challenge of germ contamination of surfaces.

Normally, control takes place by signing of a hygiene schedule. In this case, an entry (for the day and the staff member) is performed after the alleged disinfecting step.

In addition, the Verband angewandter Hygiene—VAH, Association of applied Hygiene—points out that spray disinfection in the long term is critical for staff members' health. Many floors are wiped, but work tables and cabinets are often sprayed since wipe-down disinfection is difficult due to the geometry of the equipment.

In the domestic area, dust and wipe robot systems have now been employed for a couple of years which can clean and vacuum defined surface. Since, however, they cannot move over tables or along cabinets, they are not very well suitable for the industrial area. In addition, the cost pressure here is higher than the gain in comfort of “cleaning”.

It is an object of the invention to provide an improved method and device for disinfecting clean rooms.

According to the invention, this object is achieved by a disinfecting method with the features of independent claim 1. Advantageous further developments of the disinfecting method result from the dependent claim 2 through 6. In an additional aspect, this task is solved by an unmanned aerial vehicle having the features of claim 7. Advantageous further developments of the unmanned aerial vehicle result from dependent claims 8 through 15.

### SUMMARY OF THE INVENTION

In a first aspect, the invention relates to a method of disinfecting clean rooms by administration of a disinfectant which is sprayed from the air onto the surfaces to be disinfected by means of an unmanned aerial vehicle, the disinfectant being stored on board the aerial vehicle in at least one tank.

The method according to the invention combines several decisive advantages over the method known from the state of the art.

Modern unmanned aerial vehicles can be controlled precisely and also positioned precisely in hover flight. By means of an unmanned aerial vehicle, disinfection can therefore be controlled precisely. This not only relates to the targeted approach to the surfaces but also to maintaining a defined distance from the surface which is of particular importance for effective spraying.

In this manner, the use of disinfectant can also be reduced as well as the running costs.

The use of an unmanned aerial vehicle can help to reduce staff. It must be taken into account that spray disinfection is an activity which entails health hazards for the staff. Therefore, the method according to the invention is a measure for increasing working safety.

Furthermore, man is the particle source with the greatest influence on contamination of the clean room, which is thus decisively reduced by the method according to the invention.

Unmanned aerial vehicles have by now been miniaturized to the point that they can be deposited (e. g. in a base station) on unused parking spaces (e. g. top of cabinet) in the clean room and can then directly fly through this room and disinfect it.

An unmanned aerial vehicle can reach and treat surfaces in a targeted manner which have so far been difficult to reach (ceilings, cabinet surfaces). The flying route with the spraying program can be established by previous storage of the clean room coordinates with the objects contained therein. In addition, the aerial vehicle can be equipped with distance sensors able to detect objects which are out of place, leading accordingly to an alteration of the flying route.

The control can already take place during disinfection (e. g. by imaging by means a camera).

By employing a comprehensive sensor system, other parameters indicating correct disinfection can be measured and used for controlling the disinfection process, e. g. filling-level measurement in the disinfectant tank, detection of the spraying process via sensors or detection of the current flying route.

Thus, a data set can be recorded for each disinfecting process which not only allows online control but also subsequent evaluation of correct disinfection.

Deviations in relevant parameters can be recorded and visually and/or acoustically displayed as warnings.

The existing sensor systems, together with imaging, can additionally be used for detecting critical deviations in the clean room (contaminations which have not been eliminated, rust spots, puddles etc.), and the aerial vehicle can thus also function as a “control drone”.

By using marking fluids, the flying and spraying program can in advance be tested and optimally adapted to the surfaces to be treated. This allows for the first time a thorough, cleaning process-relevant control of the disinfecting process which was not provided by the former simple signing of the hygiene schedule.

Thus, a novel validation of cleaning processes is possible as well. In a first step, an optimized flying and spraying program would be established in a controlled manner. In a second step, it would merely have to be guaranteed that this program is carried out correctly.

An unmanned drone does not need any “personal protective equipment” and can also be used in marginal periods or “downtimes”.

An aerial vehicle (which has preferably been previously parked in the clean room) reduces the infiltration and release of contaminants.

For the method according to the invention, all disinfectants which have already been established can be used. In addition, it is also possible to use disinfectants for the first time which are too toxic/incompatible for humans.

Furthermore, entirely different disinfecting methods can also be employed according to the invention, for instance release of pure gas, smoke release or a treatment with UV rays.

In addition, by upgrading the unmanned aerial vehicle, the method also allows a combination of spray disinfection with other disinfection methods, such as e. g. UV irradiation.

In summary, disinfection method according to the invention allows a clean-room disinfection which is reliable, inexpensive and quick and can be comprehensively controlled.

### DETAILED DESCRIPTION OF THE INVENTION

The invention relates to a method of disinfecting clean rooms by means of an unmanned aerial vehicle for spraying onto the surfaces to be disinfected. By storing the disinfectant on board of the aerial vehicle in a tank, an external supply (e. g. by means of a tube) can be done without, and the aerial vehicle can freely move inside the room.

In a preferred embodiment, the disinfectant is stored in the tank in undiluted form. Thus, the aerial vehicle weighs less and consumes less energy.

In one embodiment of the invention, the aerial vehicle comprises at least one device for recording proper application of the disinfectant. This device records the application advantageously online, that is, already during disinfection which takes place during the flight, and collects and/or transfers the recorded data to a control unit. This at least one recording device can detect proper application on several levels: in the tank by lowering of the filling level; in terms of spraying application, by detecting the correct spraying process and on the surface, by examining the correctly sprayed surface.

Advantageously, for detecting proper application, the device uses an imaging method preferably selected from the group consisting of photography, thermography, UV photography and reflection measurement.

In photographic imaging, the humid surface can be detected as a shining surface. Thermography can make use of the fact that disinfectants frequently contain volatile organic solvents. The evaporation enthalpy required for evaporation is taken from the environment (“evaporative cooling”), thus cooling the sprayed surface which thus can be thermographically detected. Applied UV-active disinfectants can be directly detected by UV-photography. Since wet surfaces have increased reflection, reflection measurement can be used here.

The aerial vehicle preferably contains one or more spray nozzles. By means of these nozzles, the disinfectant can be dispersed into fine droplets. Furthermore, the spraying image, and in particular the spraying angle with which the disinfectant is applied can be determined. As an alternative or supplement, it is proposed to apply the disinfectant via a valve so as to allow precise quantity control. The valve can be arranged before the spraying nozzle so as to regulate the flow to the nozzle.

In another embodiment, the at least one device for detecting correct application is adapted for detecting at least one of the following spraying parameters:
a) duration of spraying,
b) spraying amount,
c) spraying intensity,
d) spraying angle,
e) spraying surface.

Advantageously, one or more sensors are used for this purpose which are attached to the spraying device and thus can directly detect the spraying process with regard to one or more of the abovementioned spraying parameters.

The unmanned aerial vehicle according to the invention contains at least one container for storing the disinfectant. This container is advantageously a pressure tank containing a propellant gas. This entails the advantage that a pumping device can be done without and the disinfectant can be directly fed from the tank to the spraying device.

Alternatively, the storage tank can be connected to a separate pressure source. As pressure source, any device known to the person skilled in the art can be used, for instance a pressure pump or a separate pressure tank filled with propellant gas.

The aerial vehicle used in the method has at least one spraying device adapted to spray the disinfectant onto surface to be treated.

In a preferred embodiment, the spraying device is selected from the group consisting of spraying nozzle, valve, single-substance pressure nozzle such as circular-blade nozzle, turbulence nozzle or jet-forming nozzle, dual-substance nozzle with exterior mixing, dual-substance nozzle with interior mixing and rotary sprayer.

In a second aspect, the invention relates to a device for performing the disinfecting method described above, comprising at least one unmanned aerial vehicle by means of which the disinfectant can be sprayed from the air onto the surfaces to be disinfected, the aerial vehicle comprising at least one tank for storing the disinfectant.

The disinfectant tank, which is preferably a pressure tank, is preferably releasably connected to the unmanned aerial vehicle so as to be easily replaced by a new, full (pressure) tank after consumption of the disinfectant. On the aerial vehicle, a support for carrying the spray can be formed such that replacement can be performed quickly and easily.

The unmanned aerial vehicle is the central unit of the device which may be supplemented by one or more additional units. Some examples are: base unit (synonymous with “base station”), control unit, GPS unit, data exchange unit, filling unit, cleaning unit, disposal unit for used-up tanks, electrical charging unit. These units may be present as separate devices. Preferably, several units are integrated in a device. Thus, the base station may comprise an electrical charging unit, a filling unit and a control unit.

In one embodiment, the device includes a mobile GPS unit and a GPS receiver. With the mobile GPS unit, the area to be disinfected can be marked, the corresponding GPS data are received by the GPS receiver and might be used for drawing up a disinfecting schedule. Alternatively, the room to be disinfected and its objects can be measured by an ultrasonic, Bluetooth or laser-based distance meter.

The aerial vehicle according to the invention advantageously has at least one distance meter for precise flying control. This distance meter can operate with ultrasonic or laser.

The person skilled in the art is familiar with many embodiments of precisely navigating unmanned aerial vehicles. Examples of basic construction types are: duocopter, tricopter, quadrocopter, hexacopter or octocopter. These multicopters have several rotors or propellers preferably arranged on one plane and acting vertically downwards in order to create an ascending force and, by inclination of the rotor plane, a propelling force as well. Like a helicopter, the multicopter can start and land vertically.

In one embodiment, the unmanned aerial vehicle includes a camera unit for detecting correct application of the disinfectant, the camera unit allowing the performing of one or more of the following imaging methods: photography, thermography, UV photography and reflection measurement.

In another embodiment of the invention, the unmanned aerial vehicle has at least one unit for controlling one or more of the following spray parameters: spraying duration, spraying amount, spraying intensity, spraying angle, spraying surface.

In another embodiment, the aerial vehicle has an automatic shutoff system which terminates the spraying process and/or the flight when a critical event, such as, for example, the presence or approach of a person, is detected.

For application of the disinfectant, the unmanned aerial vehicle has at least one spraying unit selected from the group consisting of spraying nozzle, valve, single-substance pressure nozzle such as circular-blade nozzle, turbulence nozzle, jet-forming nozzle, dual-substance nozzle with exterior mixing, dual-substance nozzle with interior mixing and rotary sprayer.

In another embodiment, the at least one spraying device is equipped with a valve which is either controlled electronically or opens when a defined fluid pressure is exceeded.

According to one embodiment, the at least one spraying device is oriented during this process such that during spraying, it is not hindered by the air stream of the rotors. This can be achieved by attaching the spraying device on the side of the aerial vehicle which is opposite to the rotors. Consequently, it is advantageous, with rotors positioned on the top, to position the spraying device beneath the housing of the aerial vehicle. Alternatively, the aerial vehicle can have one or more screening devices which screen the spraying devices against the air stream produced by the rotor(s).

In an alternative embodiment, the spraying devices are attached such that the spraying process is supported by the air stream of the rotors. Since in the flying mode, the rotors generate an air stream directed downward, this air stream can support the spraying process and provide for accelerated drying of the disinfectant. In a preferred embodiment, spraying devices are attached centrally under the center of the rotors (that is, for instance below the rotor hub).

In a preferred embodiment, the unmanned aerial vehicle has a weight of up to 20 kg (“small UAV”) and particularly preferably a weight of up to 5 kg (“micro UAV”). A smaller aerial vehicle is easier to handle and consumes less energy.

The unmanned aerial vehicle is advantageously adapted to be used indoors and preferably inside clean rooms.

In accordance with the use in clean rooms, the unmanned aerial vehicle advantageously consists of a material which in type and surface structure is suitable for clean rooms according to DIN ISO 14644-14.

Furthermore, it is advantageous if the unmanned aerial vehicle is constructed such that areas with static air are avoided. Here, it must be taken into account that unmanned aerial vehicles are commonly operated with rotors. Therefore, the outer shape of the chassis must be adapted to the air stream generated by the rotors, with avoidance of static air.

In another embodiment of the invention, the unmanned aerial vehicle is designed such that the air stream of the rotors contacts the surface as low-turbulence unidirectional air flow.

In one embodiment of the invention, the unmanned aerial vehicle for achieving sufficient cleaning force according to DIN ISO 14644-14 has one or more of the following construction features:
a) low-joint surfaces,
b) evenness according to DIN 18 202, or better, defined absence of cracks and impermeability of the surfaces according to VDI 2083, sheet 9.1,
c) cleanability of the surfaces as specified in VDI 2083 sheet 4.1,
d) discharge capacity as specified outgassing behavior according to specification or testing on a test specimen in VDI 2083 sheet 4.1.

It is to be noted that the particle emission, which has a strong influence on contamination of the clean room, is closely linked to the surface quality of the materials employed.

In a preferred embodiment, the unmanned aerial vehicle has a housing closed on all sides. In this manner, particle release is reduced. For this purpose, it is advantageous if the drone has a heat exchanger as a cooling device. An air cooler would have the disadvantage of increased particle emission.

In a preferred embodiment, the heat exchanger is adjacent to a propellant gas containing tank; in this case, the cooling effect caused by adiabatic expansion of the gas can be used to cool the device via the heat exchanger.

In a particular embodiment, the materials suitable for clean rooms which are located in the area of movable parts of the aerial vehicle are especially relevant. The movements cause frictions between two materials, which in turn are the most frequent cause of particle emissions. By means of classification measurements and evaluations according to VDI 2083, sheet 8, the person skilled in the art can determine the cleanroom suitability of material combinations and corresponding material pairs for the respective aerial vehicle. Measurement of the friction in material testing commonly takes place by the “ball-on-disk test”, the “disk-on-disk test” or the “roller-on-disk test”.

In terms of electrostatics, in another embodiment, the surface of the aerial vehicle has an electrical field (e-field) with a sensitivity level of 4, preferably 3, especially 2 and particularly preferably of 1. The sensitivity level is defined by the intensity of an electrical field on the surface according to the following table:

| Sensitivity level | E-field intensity on the product | |
|---|---|---|
| 1 | 1 | nanocoulomb |
| 2 | 10 | nanocoulombs |
| 3 | 50 | nanocoulombs |
| 4 | 100 | nanocoulombs |

In another embodiment, the surface of the aerial vehicle has a contact resistance between 7.5×10⁵ ohm and 10⁹ ohm and a surface resistance of 10⁴ ohm/reference surface to 10¹⁰ ohm reference surface.

In a preferred embodiment, the device has means for detecting the presence of people in the clean rooms to be cleaned. This can ensure that the aerial vehicle does not collide with people present and that people do not come in contact with the disinfectant. This detecting means can be either part of the unmanned aerial vehicle or can be present in the base station or in a separate control device. This detecting means can detect the presence of people preferably via an imaging unit or a motion sensor.

In another embodiment of the invention, the device has a base station for receiving the unmanned aerial vehicle, the base station preferably being equipped for performing one or more of the following tasks:
a) electrical charging,
b) refilling of the disinfectant tank,
b) refilling of the propellant tank,
c) replacement of the disinfectant tank,
c) replacement of the propellant tank,
d) cleaning of the unmanned aerial vehicle,
e) transfer of data,
f) emergency switch-off.

In accordance with the abovementioned tasks, the base station contains one or more of the following devices:
a) electrical charging device,
b) device for filling a disinfectant tank,
c) device for replacing a disinfectant tank,
d) device for filling a propellant gas tank,
e) device for replacing a propellant gas tank,
f) data transfer device,
g) device for cleaning the aerial vehicle,
h) device for emergency switch-off.

In another aspect, the invention relates to a method of disinfecting the unmanned aerial vehicle wherein after landing (preferably in the base station), the aerial vehicle performs a thrust reversal of the rotors, with the disinfectant dispensed by the spraying device being directed upward over the housing of the aerial vehicle. In this manner, the aerial vehicle can easily be disinfected.

This method cannot only be used for disinfecting but also for cleaning the drone with spraying of a cleaning fluid. This cleaning fluid can also be dispensed by the base station.

In a preferred embodiment, the housing comprises a plastic suitable for clean rooms, or at least has a surface consisting substantially or completely of a plastic suitable for clean rooms. This plastic is preferably selected from the group consisting of polyethylene, polypropylene, polyamide, polyethylene terephthalate—PET—, polyvinylchloride, ethylene propylene diene (monomer) rubber—EPDM—and polyamide.

Advantageously, in this aerial vehicle, the materials/coatings exposed on the surface are inert with respect to the disinfectant used.

The unmanned aerial vehicle preferably has a connecting element for connection to an external filling device. This connecting element is to allow a reversible connection to the supply unit and is preferably a plug-in connector, snap-in connector, screw connector or bayonet connector.

### Definitions

An “unmanned aerial vehicle” in the framework of the invention (also called UAV, Unmanned Aerial System, AUS or colloquially drone) is an aerial vehicle which can be operated and navigated self-sufficiently by a computer or a remote control from the ground without a crew on board. Within the framework of the invention—differently from the definition of the ICAO—, this includes flying models, that is, flying devices operated in reduced or miniaturized size.

Within the framework of the application, a “clean room” is understood to be a room within which the concentration of air-borne and other contaminations stays below a defined threshold value.

A disinfection within the framework of the application is defined to be a hygiene measure designed to kill or inactivate pathogens, significantly reducing their number on an object or on a biological surface. The aim is to achieve a state in which an infection becomes improbable. The term “disinfection” is synonymous with the term “disinfection cleaning”.

Within the framework of the application, “cleanliness” is understood to be the state of a product, a surface, a device, gas, or fluid with a defined degree of contamination. A contamination is defined to be any particulate, molecular, non-particulate or biological unit which can have negative effects on the product processed in this room or on the process taking place inside the room.

A “low-turbulence unidirectional air flow (TAV)” according to the application is defined to be a controlled air flow with homogeneous speed and nearly parallel flow lines over the entire cross-section of the entire area (in accordance with the VDI guideline 2083, sheet 4.1, item 3.4.2).

The term “qualification” according to the invention is defined to be a work process for disinfection according to a defined method used for determining and/or checking the disinfection performance in a clean room or a part thereof.

According to the invention, a particulate surface cleanliness class, Oberflächenreinheitsklasse—ORK—is defined by the number of individual particles on a reference surface of 1 cm², referred to a reference particle diameter of 1 μm in a decadic evaluation system according to the VDI guideline 2083, sheet 9.1, version December 2006, FIG. 4.

In the context of the invention, a surface resistance is defined to be an electrical resistance, measured between two electrodes on this surface.

Other advantages, particularities and useful further developments of the invention can be found in the dependent Claims and in the following description of preferred embodiments by means of the Figures.

In the Figures:

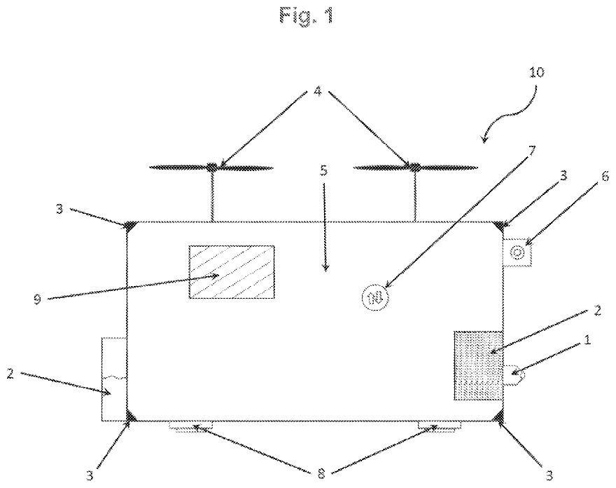

FIG. 1 is the schematic presentation of an unmanned aerial vehicle according to the invention.

### DETAILED DESCRIPTION OF EMBODIMENTS

Various embodiments of the invention are described in the following by means of the Figures.

FIG. 1 is a schematic presentation of an unmanned aerial vehicle 10 according to the invention. The aerial vehicle has an enclosure made of a material suitable for clean rooms, with a surface 5 suitable for clean rooms, and is embodied as a duocopter with two rotors 4 suitable for clean rooms. It furthermore has a spraying nozzle 1, a laterally attached tank for the disinfectant 2, sensors for indoor navigation 3, a digital camera as imaging unit 6 and a device for data transfer. Cooling takes place via a heat exchanger 9 attached to the surface. In addition, the aerial vehicle has connecting means 8 for the base station at the bottom, which means are used for electrical charging and filling.

The embodiments shown here are only examples of the present invention and are therefore not to be understood as limiting. Alternative embodiments considered by the person skilled in the art are equally comprised by the scope of protection of the present invention.

### LIST OF REFERENCE NUMBERS

10 unmanned aerial vehicle
1 spraying nozzle
2 tank for disinfectant
3 sensors for indoor navigation
4 rotors suitable for clean rooms according to DIN ISO 14644-15
5 enclosure with surface suitable for clean rooms
6 digital camera as imaging unit
7 device for data transfer
8 connecting means for base station for charging and tank filling
9 heat exchanger for cooling the aerial vehicle

1-15. (canceled)

16. Method for disinfection of clean rooms by administration of a disinfectant, the disinfectant being sprayed onto the surfaces to be disinfected from the air by means of an unmanned aerial vehicle, the disinfectant being stored in at least one tank on board the aerial vehicle, the aerial vehicle comprising at least one spraying device adapted to spray the disinfectant onto the surface to be treated and at least one device for detecting proper application of the disinfectant, which uses an imaging method selected from the group consisting of photography, thermography, UV photography and reflection measurement, wherein:
in photographic detection, a humid surface is detected as a shining surface;
in thermography, cooling of the sprayed surface is thermographically detected;
in UV photography, application of a UV-active disinfectant is directly detected by UV photography, and
in reflection measurement, a wet surface is detected by means of increased reflection.

17. Method according to claim 16, wherein at least one tank is a pressure tank containing propellant gas or is connected to a separate pressure source.

18. Method according to claim 16, wherein at least one spraying device is selected from the group consisting of spraying nozzle, valve, single-substance pressure nozzle such as circular-blade nozzle, turbulence nozzle or jet-forming nozzle, dual-substance nozzle with exterior mixing, dual-substance nozzle with interior mixing and rotary sprayer.

19. Device for performing the method according to claim 16, comprising at least one unmanned aerial vehicle by means of which the disinfectant can be sprayed from the air onto the surfaces to be disinfected, wherein the aerial vehicle contains at least one tank for storing the disinfectant and at least one spraying device adapted to spray the disinfectant onto the surface to be treated,
the unmanned aerial vehicle comprising at least one device for detecting proper application of the disinfectant, which device uses an imaging method selected from the group consisting of photography, thermography, UV photography and reflection measurement,
and the unmanned aerial vehicle containing one or more screening devices which screen the at least one spraying device against the air stream generated by the rotors.

20. Device according to claim 19, wherein the device contains at least one unit for controlling one or more of the following spraying parameters: spraying duration, spraying amount, spraying intensity, spraying angle, spraying surface.

21. Device according to claim 19, wherein the aerial vehicle has at least one spraying device selected from the group consisting of spraying nozzle, valve, single-substance pressure nozzle such as circular-blade nozzle, turbulence nozzle or jet-forming nozzle, dual-substance nozzle with exterior mixing, dual-substance nozzle with interior mixing and rotary sprayer.

22. Device according to claim 19, wherein the unmanned aerial vehicle consists of a material which is suitable for clean rooms according to DIN ISO 14644-14 in terms of the type of material and its surface structure.

23. Device according to claim 19, wherein the unmanned aerial vehicle for achieving sufficient cleanability according to DIN ISO 14644-14 has one or more of the following construction characteristics:
a) evenness, absence of cracks and impermeability of the surfaces according to VDI 2083, sheet 9.1,
b) cleanability of the surfaces as specified in VDI 2083 sheet 4.1,
c) discharge capacity as specified outgassing behavior according to specification or testing on a test specimen in VDI 2083 sheet 4.1.

24. Device according to claim 19, wherein the device contains means for detecting the presence of people in the clean rooms to be cleaned.

25. Device according to claim 19, wherein the device has a base station for receiving the unmanned aerial vehicle, the base station preferably containing one or more of the following devices:
a) Electrical charging device,
b) Device for filling a disinfectant tank,
c) Device for replacing a disinfectant tank,
d) Device for filling a propellant gas tank,
e) Device for replacing a propellant gas tank,
f) Data transfer device,
g) Device for cleaning the aerial vehicle,
h) Device for emergency switch-off.
